(19) United States
(12) Patent Application Publication
(10) Pub. No.: US 2023/0241121 A1
(43) Pub. Date: Aug. 3, 2023

(54) COMPOSITIONS AND METHODS RELATING TO EXOSOMES DERIVED FROM HUMAN DERMAL PAPILLA CELLS

(71) Applicant: NORTH CAROLINA STATE UNIVERSITY, Raleigh, NC (US)

(72) Inventors: Ke Cheng, Raleigh, NC (US); Shiqi Hu, Raleigh, NC (US)

(21) Appl. No.: 17/995,066

(22) PCT Filed: Mar. 30, 2021
(86) PCT No.: PCT/US2021/024789

§ 371 Date: Sep. 29, 2022

Related U.S. Application Data
(60) Provisional application No. 63/002,825, filed on Mar. 31, 2020.

(51) Int. Cl. A61K 35/36; C12N 5/071; C12N 15/113; A61P 17/14

(52) U.S. Cl. CPC A61K 35/36; C12N 5/0628; C12N 15/113; A61P 17/14; C12N 2513/00

(57) ABSTRACT

The present disclosure provides compositions and methods relating to the use of exosomes derived from human dermal papilla (DP) cells. In particular, the present disclosure provides novel compositions and methods for generating and maintaining exosomes derived from DP spheroids, as well as compositions and methods for delivering exosomes to a subject for various therapeutic purposes, such as the treatment of diseases and conditions related to hair follicle development.

### CROSS REFERENCE TO RELATED APPLICATION

This application claims priority to and the benefit of U.S. Provisional Patent Application No. 63/002,825 filed Mar. 31, 2020, which is incorporated herein by reference in its entirety and for all purposes.

### GOVERNMENT SUPPORT

This invention was made with government support under grant numbers HL123920, HL137093, HL144002, and HL146153 awarded by the National Institutes of Health. The government has certain rights in the invention.

### FIELD

The present disclosure provides compositions and methods relating to the use of exosomes derived from human dermal papilla (DP) cells. In particular, the present disclosure provides novel compositions and methods for generating and maintaining exosomes derived from DP spheroids, as well as compositions and methods for delivering exosomes to a subject for various therapeutic purposes, such as the treatment of diseases and conditions related to hair follicle development.

### BACKGROUND

People affected by moderate hair loss generally turn to topical treatments, like minoxidil (antihypertensive potassium channel opener) and finasteride (dihydrotestosterone-suppressing 5α-reductase inhibitor), the only Food and Drug Administration (FDA) approved treatments for inducing hair regrowth. Both typically require consistent application to maintain hair growth. Many researchers have sought a more efficient approach by focusing on the hair follicle cycle in an effort to promote a shift of the follicles from a resting phase (telogen) to an active phase (anagen). Instead of follicular unit transplantation (organ), which is costly and sometimes faces a shortage of donors, researchers have attempted to use cell therapy to treat hair loss by culturing and proliferating hair follicle cells or mesenchymal cells in vitro and then implanting them. In anagen stage, the hair follicle bulge area is an abundant source of actively growing dermal papilla cells (DPs), which drop out during resting phase. It has been suggested that hair follicles in balding areas are not disappearing, but decreasing in size. The replenishment of DPs to bald areas is, therefore, a plausible way to induce the telogen to anagen phase transition needed to induce hair growth.

The interactions between the epithelial and mesenchymal cells are vital in regulating the cycle of hair growth. As the main mesenchymal component of the follicular unit, DPs induce the cyclic changes from telogen to anagen and new hair follicles' formation. Regulating dermal papilla cells is critical in increasing cell division and the growth rate. Two-dimensional (2D) cultured DPs have demonstrated no therapeutic effect on hair follicle growth. It has been reported that three-dimensional (3D) spheroid cultures result in a partial restoration of inductive capability and are capable of inducing de novo hair follicles in human skin. DPs have to aggregate into hair follicle areas to be effective. Thus, spheroids culture therapy could be an effective way to regain the capacity for hair regeneration in vitro. However, a comprehensive understanding of the molecular mechanisms that underline this regenerative process would provide additional benefits.

### SUMMARY

Embodiments of the present disclosure include a composition comprising a plurality of exosomes derived from human dermal papilla cell (DP) spheroids. In accordance with these embodiments, the plurality of exosomes comprise at least one of (i) increased expression of at least one miRNA and/or (ii) decreased expression of at least one miRNA.

In some embodiments, the plurality of exosomes are derived from DP spheroids cultured using three-dimensional (3D) cell culture. In some embodiments, the plurality of exosomes comprise at least one of (i) and (ii) as compared to a naturally occurring DP-derived exosome. In some embodiments, the plurality of exosomes comprise at least one of (i) and (ii) as compared to a DP-derived exosome cultured using two-dimensional (2D) cell culture.

In some embodiments, the at least one miRNA is selected from the group consisting of the at least one miRNA of (i) is selected from the group consisting of miR-218-5p, let-7f-5p, let-7g-5p, let-7d-5p, miR-16-5p, let-7a-5p, miR15b-5p, miR-155-5p, miR-23b-3p, miR-22-3p, miR-125b-5p, miR-21a-5p, miR-24-3p, let-7e-5p, miR-34c-5p, miR-29a-3p, miR-93-5p, miR-125a-5p, miR-138-5p, miR-17-5p, miR-541-5p, miR-196a-5p, miR-27b-3p, let-7i-5p, miR-872-5p, let-7c-5p, miR-28c, miR-99a-5p, miR-27a-3p, miR-140-5p, miR-106b-5p, miR-9-5p, miR-23a-3p, miR-30c-5p, let-7b-5p, miR-191-5p, or any combinations thereof. In some embodiments, the at least one miRNA is enriched in the plurality of exosomes by at least 1.1-fold. In some embodiments, the at least one miRNA is selected from the group consisting of miR-218-5p, let-7f-5p, let-7g-5p, or any combinations thereof.

In some embodiments, the at least one miRNA of (ii) is selected from the group consisting of miR-31-5p, miR-29b-3p, miR-322-5p, miR-140-3p, miR-25-3p, miR-146a-5p, miR-30e-5p, miR-92a-3p, miR-20a-5p, miR-183-5p, miR-19b-3p, miR-744-5p, miR-291a-3p, miR-186-5p, miR-30a-5p, miR-199a-5p, miR-101a-3p, miR-18a-5p, miR-425-5p, miR-19a-3p, miR-126a-3p, miR-126a-5p, miR-214-3p, miR-182-5p, miR-130a-3p, miR-10a-5p, miR-30d-5p, miR-150-5p, miR-467e-5p, miR-124-3p, miR-196b-5p, miR-10b-5p, miR-15a-5p, miR-142a-3p, miR-488-3p, miR-411-5p, miR-503-5p, miR-96-5p, miR-1a-3p, miR-302d-3p, miR-467c-5p, miR-142a-5p, miR-32-5p, miR-144-3p, miR-880-3p, miR-295-3p, miR-141-3p, miR-335-5p, or any combinations thereof. In some embodiments, the at least one miRNA of (ii) is reduced in the plurality of exosomes by at least 0.9-fold. In some embodiments, the at least one miRNA of (ii) is selected from the group consisting of miR-335-5p, miR-141-3p, miR-295-3p, miR-880-3p, miR-144-3p, miR-32-5p, miR-142a-5p, or any combinations thereof.

In some embodiments, the plurality of exosomes further comprise increased expression of at least one hair follicle regulatory gene. In some embodiments, the at least one hair follicle regulatory gene is selected from FGF2, TIMP2, or a combination thereof.

Embodiments of the present disclosure also includes a method of generating a plurality of exosomes capable of modulating at least one characteristic of skin tissue. In accordance with these embodiments, the method includes culturing human dermal papilla cell (DP) spheroids using three-dimensional (3D) cell culture, and isolating a plurality of exosomes from the DP spheroids. In some embodiments, the plurality of exosomes comprise at least one of (i) increased expression of at least one miRNA, and/or (ii) decreased expression of at least one miRNA. In some embodiments, the plurality of exosomes comprise at least one of (i) and (ii) as compared to a naturally occurring DP-derived exosome. In some embodiments, the plurality of exosomes have at least one of (i) and (ii) as compared to an DP-derived exosome cultured using two-dimensional (2D) cell culture.

Embodiments of the present disclosure also include a method of treating a skin condition or disease. In accordance with these embodiments, the method includes administering a plurality of exosomes derived from dermal papilla cell (DP) spheroids to a subject in need thereof. In some embodiments, administering the plurality of exosomes modulates at least one characteristic of the subject's skin tissue. In some embodiments, the plurality of exosomes comprise at least one of (i) increased expression of at least one miRNA, and/or (ii) decreased expression of at least one miRNA. In some embodiments, the plurality of exosomes are derived from DP spheroids cultured using three-dimensional (3D) cell culture. In some embodiments, the plurality of exosomes comprise at least one of (i) and (ii) as compared to a naturally occurring DP-derived exosome. In some embodiments, the plurality of exosomes comprise at least one of (i) and (ii) as compared to an DP-derived exosome cultured using two-dimensional (2D) cell culture.

In some embodiments, the plurality of exosomes are administered to the subject's skin via injection, microinjection (microneedles), intradermal (ID) injection, subcutaneous (SC) injection, a non-invasive method, needle-free injection, or topical application.

In some embodiments, modulating at least one characteristic of the subject's skin tissue comprises at least one of (i) increasing expression of β-catenin; (ii) decreasing expression of SFRP2; (iii) enhancing DP cell migration and/or survival; (iv) enhancing hair follicle growth; and/or (v) maintaining anagen phase of hair cycle. In some embodiments, modulating at least one of (i)-(v) treats the subject's skin condition or disease. In some embodiments, the skin condition or disease comprises male or female-pattern hair loss, alopecia areata, telogen effluvium or anagen effluvium.

### BRIEF DESCRIPTION OF THE DRAWINGS

FIGS. 1A-1I: The preparation and characterization of 3D DP spheroids. (A) The isolation of mouse dermal papilla cells (DPs) from vibrissae. Scale bar: 500 μm. (B) Conventional culture enables the growth of 2D DPs. Scale bar: 100 μm. (C) Growth of DP spheroids in ultra-low cell culture flasks. Scale bar: 100 μm. (D) Double staining of CD133 (green) and β-catenin (red) of spheroids. (E-F) Scanning Electron Microscope (SEM) images of keratin (E) and 3D spheroid-loaded keratin. (F) One obvious spheroid is highlighted in yellow. (G) Schematic illustrating the injection sites on the back of a mouse for the cell retention study. (H) The mouse was shaved and injected on the dorsal skin with different formulations as illustrated in (G) on the dorsal skin. Cells were labeled by DiD and then resuspended in PBS or keratin for intradermal injection. In vivo imaging system (IVIS) images were taken at different time points. (I) Quantification of IVIS images. Data shown as mean±S.D., n=3 mic). 3D spheroids/keratin showed the longest retention time.

FIGS. 2A-2G: Comparison of the hair follicle phase with topical treatment of 5% minoxidil against injecting 2D DP cells or 3D spheroids, respectively. (A) Illustration of the treatment side (left) and remote side (right) of a depilated mouse. (B) The expression of β-catenin after different treatments at both the treated site and remote site. (C) Representative images showing the expression of CD133 after different treatments at both the treated site and remote site. (D) The expression of Ki67 after different treatments at both the treated site and remote site. (E-G) Quantification of β-catenin positive (β-catenin^{pos}) (E), CD133^{pos}(F) and Ki67^{pos} cells (G). Pink indicates the treated site and grey indicates the remote site. n=5, n.s. means no significant difference, * P<0.05, ** P<0.01, ***P<0.001.

FIGS. 3A-3B: Dorsal hair growth experiment on C57BL/6 mice. (A) Observation of hair coverage. Mice were divided into five groups (n=5) and treated on their left halves. Mice were imaged on days 0, 10, 15 and 20 respectively. (B) Quantification of hair coverage on Day 10, 15 and 20. Both the left and right sites were recorded. n=5, n.s. means no significant difference, * P<0.05, ** P<0.01, ***P<0.001.

FIGS. 4A-4E: Western blotting and immunohistology with various treatments. (A) Western blot of p-catenin, p-Erk ½, Erk ½, SFRP2 and GAPDH protein content in the dorsal skin on Day 20. B) Quantification of Western blot protein levels by group. n=3, n.s., no significant difference, * p<0.05, ** p<0.01, ***p<0.001. (C) Immunofluorescent co-staining of SFRP2 and β-catenin of the skin samples from different groups. Nuclei were stained by 4′,6-Diamidino-2-Phenylindole, Dihydrochloride (DAPI, blue). Scale bar: 50 μm. (D) Quantification of the relative expression of SFRP2. (E) Quantification of relative expression of β-catenin. n=5, n.s. means no significant difference, * P<0.05, ** P<0.01, ***P<0.001.

FIGS. 5A-5E: Secretome from 2D DPs and 3D DP spheroids. (A) Schematic illustrating how DP spheroids promote the hair cycle transition from catagen to telogen via the migration and secretion of factors and exosomes. In anagen, an abundant source of growing DPs is inside the follicle bulge. DPs drop during catagen. The replenishment of DPs promotes the onset of anagen. (B-E) Characterization of 2D DP-XOs and 3D DP-XOs. (B) TEM images. Exosomes were pointed out by yellow arrows. Scale bar: 500 μm. (C) Size distribution by NanoSight. (D) Top ten up-regulated and ten down-regulated miRNAs of 3D DP-XOs compared to 2D DP-XOs. (n=3 biological replicates, 3 technical replicates for each biological replicate). (E) Schematic illustrating FGF2- and TIMP2-driven hair follicle regulation and miR-218-5p-enduced promotion of hair follicle development.

FIGS. 6A-6G: Effects of exosome treatment on dorsal hair regrowth. (A) Mice were divided into three groups (n=4) and treated on their left sides. Mice were imaged on days 10 and 15 respectively. (B) Corresponding hair coverage analysis of different groups. Both the left and right sides were recorded. 3D DP-XOs promote hair follicle growth more effectively than minoxidil and 2D DP-XOs. n=4, * P<0.05, ** P<0.01, ***P<0.001, **** P<0.0001. (C) Immunofluorescent co-staining of SFRP2 (green) and β-catenin (red) on skin samples from different treatment groups. Nuclei were stained by DAPI (blue). Scale bar: 100 μm. (D) Quantification of the relative expression of SFRP2. n=5, ***P<0.001. (E) Representative mice imaged on Day 20 after injection of negative control, miR-218-5p mimics and inhibitors, respectively. Red circles indicate injection spots. There is a small bald spot on the injection site, which means that the delivery approach needs further modification. (F) Quantification of hair coverage level (%) of three groups on day 15. n=4, * P<0.05, ** P<0.01. (G) miR-218-5p enriched exosomes and miR-218-5p mimics delivered via in vivo jetPEI can transfect miR-218-5p to target SFRP2, and thus upregulate Wnt/β-catenin pathway; miR-218-5p will block this signaling to a certain degree.

FIGS. 7A-7B: Size distribution of DP spheroids. (A) Four representative images of DP spheroids in ultra-low attachment 96-well plates. Scale bar: 200 μm. (B) Size quantification of the spheroids.

FIGS. 8A-8B: Immunocytochemistry of 2D DPs. The expressions of β-catenin (A) and CD133 (B) of DPs were confirmed via confocal imaging. Scale bar: 100 μm.

FIGS. 9A-9B: CD133 and β-catenin expressions on 2D DPs and 3D DP spheroids. Flow cytometry showed the expression of CD133 and β-catenin of 2D DPs (A) and spheroids (B).

FIG. 10: Quantification of DiD^{pos} cells in the injected site and the remote site. n=5, ** P<0.01, ****P<0.0001.

FIGS. 11A-11D: Masson's trichrome and Haemotoxylin and Eosin (H&E) staining of dorsal skin after treatment. (A) Representative Masson's trichrome staining and (B) H&E staining images of each group (scale bar: 200 μm). (C) Dorsal skin with thinnest fur of each group was collected and stained. Masson's trichrome staining (scale bar: 200 μm). (D) Dorsal skin with thickest fur of each group was collected and stained. H&E staining (scale bar: 60 μm). Collagen layers were labeled by yellow arrows. Follicles were pointed out by red arrows in (D). From left to right: PBS (intradermal), 5% minoxidil, keratin, 3D spheroids (PBS), and 3D spheroids (keratin).

FIG. 12: Cytokine array reveals the difference between factors secreted from 2D DPs and 3D DP spheroids. n=3, ** P<0.01, ****P<0.0001.

FIG. 13: Western blot of common exosomal markers, Alix, CD9 and CD81.

FIG. 14: miRNA array analysis of 3D DP-XOs and 2D DP-XOs. (A) Representative miRNA array showed the differences in miRNA expression between 3D DP-XOs from 2D DP-XOs. (B) Fold difference (3D/2D DP-XOs) of mmu-miRNAs. (n=3 biological replicates, 3 technical replicates for each biological replicate).

FIGS. 15A-15B: Effects of 2D DP-XOs and 3D DP spheroid-XOs on cell migration. (A) Cell images on 0 h, 12 h and 24 h with the incubation with exosomes. Scale bar: 50 μm. (B) Quantification of cells that migrated in the gap area. n=3, * P<0.05, ****P<0.0001.

FIG. 16: Immunohistochemistry of miR-218 mimic or inhibitor treated skin samples. Immunofluorescent co-staining of SFRP2 (green) and β-catenin (red) of the skin from different groups. Nuclei were stained by DAPI (blue). Scale bar: 50 μm.

### DETAILED DESCRIPTION

Embodiments of the present disclosure provide novel compositions and methods for generating and maintaining exosomes derived from dermal papilla cell spheroids. As described further herein, the progression of the hair follicle cycle from the telogen to the anagen phase is the key to regulating hair regrowth. Dermal papilla cells (DPs) support hair growth and regulate hair cycling. However, they gradually lose key inductive properties upon culture. DPs can partially restore their capacity of promoting hair regrowth with spheroid culture. In the present disclosure, it was found that DP spheroids are effective in promoting the hair follicle cycle from telogen to anagen (e.g., compared to DPs or 5% minoxidil). Due to the importance of paracrine signaling in this process, secretome and exosomes were isolated from DP cell culture and their therapeutic efficacies were compared. Embodiments of the present disclosure also demonstrated that miR-218-5p was notably upregulated in DP spheroid-derived exosomes, and that DP spheroid-derived exosomes downregulated the secreted frizzled related protein 2 (SFRP2) and upregulated O-catenin while promoting the development and maintenance of hair follicles.

The differentially expressed secretome or exosomes from DP spheroids and DPs could be important for regulating hair follicle cycles. Recently, DP-derived secretome and extracellular vesicles demonstrated an effect in promoting hair growth, but the mechanism is not yet understood. Compared to mesenchymal stem cell derived exosomes, DP-derived exosomes were demonstrated to be efficient TGF-β activators and proven to be important in promoting proliferation of human hair follicle dermal papilla cells and hair growth. Three-dimensional (3D) culture has been proven to be a way to enrich certain proteins and miRNAs in secretome. Previous studies have demonstrated that exosomes derived from 3D DP (3D DP-XOs) promoted the proliferation of DP cells and outer root sheath cells and increased the expression of growth factors in DP cells. However, the importance of differentially expressed secretome and miRNAs from 2D DPs and 3D DPs and possible mechanisms of 3D DPs or 3D DP-XOs in promoting hair regeneration have not yet been demonstrated.

Given the limitations of current approaches for treating hair loss, such as the temporary efficacy of finasteride and minoxidil and the limited number of treatments available, the need to discover new therapies for preventing hair loss and enhancing hair regrowth remains an urgent and unmet need. Although replenishing DPs cells with in vitro-cultured DPs is one possible approach for driving the telogen to anagen transition in the hair follicle cycle, DPs will lose their hair-inducing capacity over time when they are cultured on a flat, plastic surface. For example, it has been observed that cultured DP cells (e.g., over 6 passages) could not induce any hair follicles to transition to anagen after implantation in situ. DPs have to condensate in the hair follicle bulb to promote folliculogenesis. Embodiments of the present disclosure demonstrated a higher expression of CD133 and β-catenin of DP spheroids compared to DPs, which indicated that DP spheroids could exhibit better hair growth induce capacity in vivo.

Secretome from DP spheroids is also different from that of dissociated DP cells. Researchers have shown that dermal spheres are morphologically akin to DPs in the anagen phase with expression profiles different from 2D cells but greatly similar to intact DPs. Evidence of how the transplanted hair follicles affect the environment of the bald area and a paracrine effect was not sufficient. Embodiments of the present disclosure demonstrated that DPs exerted their regulatory effects on hair cycles mainly through a paracrine mechanism. The expression of β-catenin was upregulated not only in the injected sites but also in remote sites. DPs release various growth factors and vesicles to regulate follicular biology, and it was established that cultivation in spheroids most efficiently preserves the initial phenotype of these cell in vitro. Rather than introducing more DPs to accumulate into hair follicles, exosomes were injected to regulate hair follicle cycles. Spheroid-derived exosomes also enhanced the expression of β-catenin and downregulated SFRP2, which positively regulated hair follicle growth and maintain the anagen phase of hair cycle.

Although C57BL/6 mice are useful models for screening agents promoting hair growth, as their skin pigmentation is producing pigment only during anagen. Hormonal, environmental, and/or genetic causes of hair loss are more applicable to humans and other mammals. Cell or cell secretome therapy may offer certain advantages in treating those types of hair loss compared to minoxidil, since the mechanism of minoxidil is to increase the cutaneous blood flow to the treated site and cause hyperpolarization of cell membranes, allowing more nutrients to reach the follicles and cells. Embodiments of the present disclosure have demonstrated that the spheroids promoted the hair cycle from catagen to anagen in healthy mice. Disease-related models or hormone-related hair loss models will likely benefit equally from the enhanced therapeutic effects of exosome therapy, as opposed to minoxidil.

As described further herein, results of the present disclosure have demonstrated that exosomes enriched with certain miRNAs and regulatory genes (e.g., miR-218-5p and TIMP2) accelerated the onset of anagen, and 3D spheroid cultures provided a valuable mechanism for cell therapy based on exosome delivery. As provided herein, miR-218-5p regulated hair follicle development by down-regulating Wnt signaling inhibitors SFRP2, thus promoting O-catenin, creating a positive feedback loop promoting hair development and regrowth. The administration of therapy based on these factors (e.g., exosomes derived from DP spheroids) offers a minimally invasive alternative approach with advantages over current therapeutic approaches to hair loss.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like.

The term “cell culture process” or “cell culture” generally refers to the process by which cells are grown or maintained under controlled conditions. The cell culture process may take place in vitro or ex vivo. In some embodiments, a cell culture process has both an expansion phase and a production phase. In some embodiments, the expansion and production phases are separated by a transition or shift phase. “Culturing” a cell refers to contacting a cell with a cell culture medium under conditions suitable to for growing or maintaining the cell. A “cell culture” can also refer to a solution containing cells. In some embodiments, cell cultures can be three-dimensional (3D) or two-dimensional (2D). Generally, 2D cell culture systems grow cells on flat dishes, typically made of plastic. The cells are put onto coated surfaces where they adhere and spread in a two-dimensional fashion. Generally, 3D cell culture systems can be described as the culture of living cells within micro-assembled devices and supports that provide a three-dimensional structure mimicking tissue and organ specific microarchitecture (see e.g., John W. Haycock et al. “3D Cell Culture: A Review of Current Approaches and Techniques”).

The terms “medium” and “cell culture medium” (plural, “media”) generally refer to a nutrient source used for growing or maintaining cells. As is understood by a person of ordinary skill in the art, a growth medium or cell culture medium is a liquid or gel designed to support the growth of microorganisms, cells, or small plants. Cell culture media generally comprise an appropriate source of energy and compounds which regulate the cell cycle. A typical culture medium can be composed of, but not limited to, a complement of amino acids, vitamins, inorganic salts, glucose, and serum as a source of growth factors, hormones, and attachment factors. In addition to nutrients, the medium also helps maintain pH and osmolality.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the compositions of the present disclosure encompass any composition made by admixing a component of the present disclosure (e.g., exosomes derived from human dermal papilla cell (DP) spheroids) and a pharmaceutically acceptable carrier and/or excipient. When a component of the present disclosure is used contemporaneously with one or more other drugs, a composition containing such other drugs in addition to a component of the present disclosure is contemplated. Accordingly, the compositions of the present disclosure include those that also contain one or more other active ingredients, in addition to a component of the present disclosure. The weight ratio of a component of the present disclosure to the second active ingredient may be varied and will depend upon the effective dose of each ingredient. Generally, an effective dose of each will be used. Combinations of a component of the present disclosure and other active ingredients will generally also be within the aforementioned range, but in each case, an effective dose of each active ingredient should be used. In such combinations, a component of the present disclosure and other active agents may be administered separately or in conjunction. In addition, the administration of one component may be prior to, concurrent to, or subsequent to the administration of other agent(s).

The term “pharmaceutical composition” as used herein refers to a composition that can be administered to a subject to treat or prevent a disease or pathological condition in the patient (e.g., exosomes derived from human dermal papilla cell (DP) spheroids). The compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

Formulations suitable for administration include, for example, aqueous sterile injection solutions, which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (e.g., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell (e.g., extracellular vesicle, exosome, liposome, etc.) obtained from a tissue or cell. The term “derived from” may also refer to a component or fragment of a cell from a tissue or cell, including, but not limited to, a protein, a nucleic acid, a membrane or fragment of a membrane, and the like.

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

The terms “administration of” and “administering” a composition as used herein refers to providing a composition of the present disclosure to a subject in need of treatment (e.g., treatment of diseases and conditions related to hair follicle development). The compositions of the present disclosure may be administered by oral, parenteral (e.g., intramuscular, intraperitoneal, intravenous, ICV, intracisternal injection or infusion, subcutaneous injection, nebulization, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. COMPOSITIONS AND METHODS

The present disclosure provides compositions and methods relating to the use of exosomes derived from human dermal papilla (DP) cells. In particular, the present disclosure provides novel compositions and methods for generating and maintaining exosomes derived from DP spheroids, as well as compositions and methods for delivering exosomes to a subject for various therapeutic purposes, such as the treatment of diseases and conditions related to hair follicle development.

Embodiments of the present disclosure include a composition comprising a plurality of exosomes derived from human dermal papilla cell (DP) spheroids. In accordance with these embodiments, the plurality of exosomes comprise various factors that enhance hair follicle development and promote hair growth. In some embodiments, the plurality of exosomes are derived from DP spheroids cultured using three-dimensional (3D) cell culture. In some embodiments, the plurality of exosomes are enriched with various factors (e.g., miRNAs and/or hair follicle regulatory genes) as compared to a naturally occurring DP-derived exosome, or compared to a DP-derived exosome cultured using two-dimensional (2D) cell culture. The DP-derived exosomes from 3D cell cultures can be enriched for various factors due to increased expression, abundance, degree of activity or efficacy, and/or any combination thereof.

In some embodiments, the exosomes of the present disclosure exhibit increased expression of at least one miRNA. In some embodiments, the exosomes are enriched for or have increased expression of miR-218-5p, let-7f-5p, let-7g-5p, let-7d-5p, miR-16-5p, let-7a-5p, miR15b-5p, miR-155-5p, miR-23b-3p, miR-22-3p, miR-125b-5p, miR-21a-5p, miR-24-3p, let-7e-5p, miR-34c-5p, miR-29a-3p, miR-93-5p, miR-125a-5p, miR-138-5p, miR-17-5p, miR-541-5p, miR-196a-5p, miR-27b-3p, let-7i-5p, miR-872-5p, let-7c-5p, miR-28c, miR-99a-5p, miR-27a-3p, miR-140-5p, miR-106b-5p, miR-9-5p, miR-23a-3p, miR-30c-5p, let-7b-5p, miR-191-5p, or any combination thereof. In some embodiments, the exosomes are enriched for or have increased expression of miR-218-5p, let-7f-5p, and let-7g-5p, or any combination thereof. In some embodiments, the miRNA is more highly expressed or in higher abundance by at least 1.1-fold as compared to naturally occurring exosomes or exosomes from 2D cell cultures. In some embodiments, the miRNA is at least 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 4.5-fold, 5.0-fold, 5.5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, or more highly expressed or in higher abundance as compared to naturally occurring exosomes or exosomes from 2D cell cultures. In some embodiments, the exosomes have increased expression of miR-218-5p by at least 25-fold.

In some embodiments, the exosomes of the present disclosure exhibit decreased expression of at least one miRNA. In some embodiments, the exosomes are reduced for or have decreased expression of miR-31-5p, miR-29b-3p, miR-322-5p, miR-140-3p, miR-25-3p, miR-146a-5p, miR-30e-5p, miR-92a-3p, miR-20a-5p, miR-183-5p, miR-19b-3p, miR-744-5p, miR-291a-3p, miR-186-5p, miR-30a-5p, miR-199a-5p, miR-101a-3p, miR-18a-5p, miR-425-5p, miR-19a-3p, miR-126a-3p, miR-126a-5p, miR-214-3p, miR-182-5p, miR-130a-3p, miR-10a-5p, miR-30d-5p, miR-150-5p, miR-467e-5p, miR-124-3p, miR-196b-5p, miR-10b-5p, miR-15a-5p, miR-142a-3p, miR-488-3p, miR-411-5p, miR-503-5p, miR-96-5p, miR-1a-3p, miR-302d-3p, miR-467c-5p, miR-142a-5p, miR-32-5p, miR-144-3p, miR-880-3p, miR-295-3p, miR-141-3p, miR-335-5p, or any combinations thereof. In some embodiments, the exosomes are reduced for or have decreased expression of miR-335-5p, miR-141-3p, miR-295-3p, miR-880-3p, miR-144-3p, miR-32-5p, miR-142a-5p, or any combinations thereof. In some embodiments, the miRNA has decreased expressed or is present in lower abundance by at least 0.9-fold as compared to naturally occurring exosomes or exosomes from 2D cell cultures. In some embodiments, the miRNA is at least 0.8-fold, 0.7-fold, 0.6-fold, 0.5-fold, 0.45-fold, 0.4-fold, 0.35-fold, 0.3-fold, 0.25-fold, 0.2-fold, 0.15-fold, 0.1-fold, 0.09-fold, 0.08-fold, 0.07-fold, 0.06-fold, 0.05-fold, 0.04-fold, 0.03-fold, 0.02-fold, 0.01-fold, or less expressed or present in lower abundance as compared to naturally occurring exosomes or exosomes from 2D cell cultures.

In some embodiments, the exosomes of the present disclosure exhibit increased expression of at least one hair follicle regulatory gene. In some embodiments, the exosomes are enriched for or have increased expression of FGF2, TIMP2, or a combination thereof. In some embodiments, the exosomes are enriched for or have increased expression of CD133. In some embodiments, the hair follicle regulatory gene is more highly expressed or in higher abundance by at least 5-fold as compared to naturally occurring exosomes or exosomes from 2D cell cultures. In some embodiments, the hair follicle regulatory gene is at least 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, or more highly expressed or in higher abundance as compared to naturally occurring exosomes or exosomes from 2D cell cultures. In some embodiments, the exosomes have increased expression of any factor that is a positive regulator of FGF signaling and/or Wnt/β-catenin signaling, including but not limited to FGF2 and TIMP2.

Embodiments of the present disclosure also includes a method of generating a plurality of exosomes capable of modulating at least one characteristic of skin tissue. In accordance with these embodiments, the method includes culturing human dermal papilla cell (DP) spheroids using three-dimensional (3D) cell culture, and isolating a plurality of exosomes from the DP spheroids. In some embodiments, the plurality of exosomes are enriched with various factors (e.g., miRNAs and/or hair follicle regulatory genes) as compared to a naturally occurring DP-derived exosome, or compared to a DP-derived exosome cultured using two-dimensional (2D) cell culture. In some embodiments, the method includes generating exosomes that have increased expression of or are enriched for miR-218-5p, let-7f-5p, let-7g-5p, let-7d-5p, miR-16-5p, let-7a-5p, miR15b-5p, miR-155-5p, miR-23b-3p, miR-22-3p, miR-125b-5p, miR-21a-5p, miR-24-3p, let-7e-5p, miR-34c-5p, miR-29a-3p, miR-93-5p, miR-125a-5p, miR-138-5p, miR-17-5p, miR-541-5p, miR-196a-5p, miR-27b-3p, let-7i-5p, miR-872-5p, let-7c-5p, miR-28c, miR-99a-5p, miR-27a-3p, miR-140-5p, miR-106b-5p, miR-9-5p, miR-23a-3p, miR-30c-5p, let-7b-5p, miR-191-5p, or any combinations thereof. In some embodiments, the plurality of exosomes have reduced amounts of various factors (e.g., miRNAs and/or hair follicle regulatory genes) as compared to a naturally occurring DP-derived exosome, or compared to a DP-derived exosome cultured using two-dimensional (2D) cell culture. In some embodiments, the method includes generating exosomes that have decreased expression of miR-31-5p, miR-29b-3p, miR-322-5p, miR-140-3p, miR-25-3p, miR-146a-5p, miR-30e-5p, miR-92a-3p, miR-20a-5p, miR-183-5p, miR-19b-3p, miR-744-5p, miR-291a-3p, miR-186-5p, miR-30a-5p, miR-199a-5p, miR-101a-3p, miR-18a-5p, miR-425-5p, miR-19a-3p, miR-126a-3p, miR-126a-5p, miR-214-3p, miR-182-5p, miR-130a-3p, miR-10a-5p, miR-30d-5p, miR-150-5p, miR-467e-5p, miR-124-3p, miR-196b-5p, miR-10b-5p, miR-15a-5p, miR-142a-3p, miR-488-3p, miR-411-5p, miR-503-5p, miR-96-5p, miR-1a-3p, miR-302d-3p, miR-467c-5p, miR-142a-5p, miR-32-5p, miR-144-3p, miR-880-3p, miR-295-3p, miR-141-3p, miR-335-5p, or any combinations thereof. In some embodiments, the method includes generating exosomes that have increased expression of or are enriched for the at least one hair follicle regulator gene, such as FGF2 and/or TIMP2.

Embodiments of the present disclosure also include a method of treating a skin condition or disease. In accordance with these embodiments, the method includes administering a plurality of exosomes derived from dermal papilla cell (DP) spheroids to a subject in need thereof. In some embodiments, administering the plurality of exosomes modulates at least one characteristic of the subject's skin tissue. In some embodiments, the plurality of exosomes are enriched with various factors (e.g., miRNAs and/or hair follicle regulatory genes) as compared to a naturally occurring DP-derived exosome, or compared to a DP-derived exosome cultured using two-dimensional (2D) cell culture. In some embodiments, the method includes generating exosomes that have increased expression of or are enriched for miR-218-5p, let-7f-5p, let-7g-5p, let-7d-5p, miR-16-5p, let-7a-5p, miR15b-5p, miR-155-5p, miR-23b-3p, miR-22-3p, miR-125b-5p, miR-21a-5p, miR-24-3p, let-7e-5p, miR-34c-5p, miR-29a-3p, miR-93-5p, miR-125a-5p, miR-138-5p, miR-17-5p, miR-541-5p, miR-196a-5p, miR-27b-3p, let-7i-5p, miR-872-5p, let-7c-5p, miR-28c, miR-99a-5p, miR-27a-3p, miR-140-5p, miR-106b-5p, miR-9-5p, miR-23a-3p, miR-30c-5p, let-7b-5p, miR-191-5p, or any combinations thereof. In some embodiments, the plurality of exosomes have reduced amounts of various factors (e.g., miRNAs and/or hair follicle regulatory genes) as compared to a naturally occurring DP-derived exosome, or compared to a DP-derived exosome cultured using two-dimensional (2D) cell culture. In some embodiments, the method includes generating exosomes that have decreased expression of miR-31-5p, miR-29b-3p, miR-322-5p, miR-140-3p, miR-25-3p, miR-146a-5p, miR-30e-5p, miR-92a-3p, miR-20a-5p, miR-183-5p, miR-19b-3p, miR-744-5p, miR-291a-3p, miR-186-5p, miR-30a-5p, miR-199a-5p, miR-101a-3p, miR-18a-5p, miR-425-5p, miR-19a-3p, miR-126a-3p, miR-126a-5p, miR-214-3p, miR-182-5p, miR-130a-3p, miR-10a-5p, miR-30d-5p, miR-150-5p, miR-467e-5p, miR-124-3p, miR-196b-5p, miR-10b-5p, miR-15a-5p, miR-142a-3p, miR-488-3p, miR-411-5p, miR-503-5p, miR-96-5p, miR-1a-3p, miR-302d-3p, miR-467c-5p, miR-142a-5p, miR-32-5p, miR-144-3p, miR-880-3p, miR-295-3p, miR-141-3p, miR-335-5p, or any combinations thereof. In some embodiments, the method includes generating exosomes that have increased expression of or are enriched for the at least one hair follicle regulator gene, such as FGF2 and/or TIMP2. In some embodiments, the plurality of exosomes are administered to the subject's skin via injection, microinjection (microneedles), intradermal (ID) injection, subcutaneous (SC) injection, a non-invasive method, needle-free injection, or topical application.

In some embodiments, modulating at least one characteristic of the subject's skin tissue includes increasing expression of β-catenin, decreasing expression of SFRP2, enhancing DP cell migration and/or survival, enhancing hair follicle growth, and/or maintaining anagen phase of hair cycle. In some embodiments, modulating any one or more of these aspects treats the subject's skin condition or disease. In some embodiments, the skin condition or disease comprises male or female-pattern hair loss, alopecia areata, telogen effluvium or anagen effluvium.

As would be recognized by one of ordinary skill in the art based on the present disclosure, the various factors disclosed herein (e.g., miRNAs and/or hair follicle regulatory genes) that are enriched in DP spheroid-derived exosomes as compared to a naturally occurring DP-derived exosome or a DP-derived exosome cultured using two-dimensional (2D) cell culture can be included in a therapeutic composition and used to treat a hair loss condition with or without the presence of an exosome or spheroid, and can be delivered to a subject using means known in the art (e.g., targeted gene delivery). In some embodiments, these factors can also be included in a therapeutic composition that is comprised of synthetically engineered exosomes or spheroids, or other similar delivery vesicles known in the art.

In some embodiments, the compositions of the present disclosure comprise at least one pharmaceutically acceptable excipient or carrier. A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising extracellular vesicles (e.g., exosomes), a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered extracellular vesicle (e.g., exosome) and any corresponding cargo (e.g., microRNA). Adjusting the dose to achieve maximal efficacy based on the methods described above and other methods as are well-known in the art is well within the capabilities of the ordinarily skilled person in the art.

A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising extracellular vesicles (e.g., exosomes), a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered extracellular vesicles and any corresponding cargo (e.g., microRNA). Adjusting the dose to achieve maximal efficacy based on the methods described above and other methods as are well-known in the art is well within the capabilities of the ordinarily skilled person in the art.

The various compositions of the present disclosure provide dosage forms, formulations, and methods that confer advantages and/or beneficial pharmacokinetic profiles. A composition of the disclosure can be utilized in dosage forms in pure or substantially pure form, in the form of its pharmaceutically acceptable salts, and also in other forms including anhydrous or hydrated forms. A beneficial pharmacokinetic profile may be obtained by administering a formulation or dosage form suitable for once, twice a day, or three times a day, or more administration comprising one or more composition of the disclosure present in an amount sufficient to provide the required concentration or dose of the composition to an environment of use to treat a disease disclosed herein, in particular a cancer.

A medicament or treatment of the disclosure may comprise a unit dosage of at least one composition of the disclosure to provide therapeutic effects. A “unit dosage or “dosage unit” refers to a unitary (e.g., a single dose), which is capable of being administered to a patient, and which may be readily handled and packed, remaining as a physically and chemically stable unit dose comprising either the active agents as such or a mixture with one or more solid or liquid pharmaceutical excipients, carriers, or vehicles.

### 3. MATERIALS AND METHODS

Isolation of dermal papillae cells from C57BL 6 mice. The vibrissa pads were cut off from a euthanized C57BL/6 mouse and then rinsed in PBS three times. Hair follicles were dissected and incubated with 0.25% dispase (STEMCELL Technologies) for 20 minutes in the incubator. Then, a horizontal cut above the dermal papilla was made. Then, the hair follicle bulbs were transferred into a rat-tail collagen I (Sigma-Aldrich) coated dish. Eagle's minimal essential medium (MEM; Gibco), 10% FBS (Corning), 1% penicillin-streptomycin and 10 ng/ml bFGF (Fisher Scientific) was used as DP media. DPs grew out from bulbs about three days later. Then, hair follicle bulbs were washed off and placed in fresh media. DPs reached confluence after one week.

2D cell and 3D spheroid culture. For 2D culture, rat-tail collagen I coated flasks were used for passage. Passage 3-6 DPs were used. For 3D culture, DPs were seeded in an ultra-low attachment 96-well plate (Corning, 5×10⁴ per well) to count the number spheroids. Spheroids were formed on day 2 after seeding. Day 5-7 spheroids were used in animal study. DP media (MEM, 10% FBS, 1% penicillin-streptomycin and 10 ng/ml bFGF) was used for cell culture. To collect conditioned media or exosomes, the DP media was changed to basic MEM media (no FBS) once cells reached 80% confluence. Then conditioned media was collected (three days).

Secretome and exosome isolation and analysis. Conditioned media was concentrated via Amicon® Ultra-15 centrifugal filter units (3 KDa cut-off) and washed with PBS once. Exosomes were concentrated via Ultra-15 centrifugal filter units (100 KDa cut-off) and washed with PBS once. Mouse Angiogenesis Array (Raybio) and miRNA PCR Array Mouse miFinder (Qiagen) were used according to the manufacturer's instruction.

Preparation of keratin hydrogels. Keratin hydrogel was prepared according to previously publication with modifications. Briefly, hair (local barber) was washed with water and then cut into small pieces. Then, hair fragments were treated with 2.5% (w/v) peracetic acid overnight before being washed thoroughly with running water. Hair fragments were immersed in 150 mM Tris base for two hours to extract proteins. Then, clear solution was obtained after fibers removed via a 40 μm sieve, purified by dialysis against DI water (with 3 KDa molecular cut-off dialysis membrane, Fisher Scientific), and freeze-dried (lyophilized). The resulting lyophilized solid was further ground into a fine powder. Fifteen percent keratin hydrogels (w/v) were prepared by reconstituting the fine powder with PBS. Spheroids were dispersed in keratin solution and left in an incubator overnight while the keratin formed a hydrogel.

Scanning electron microscopy (SEM). The morphology of the hydrogel was visualized via SEM. Samples were fixed with 2% glutaraldehyde, and then dehydrated in gradient ethanol successively for 10 min each. They were then dried in hexamethyldisilazane (Sigma-Aldrich). Gels were sputter-coated with gold before imaging (JEOL 6010LA SEM, JEOL ltd, Japan).

Animals and in vivo studies. Seven-week-old male C57BL/6 mice were purchased from Jackson laboratory and allowed to adapt to their new environment for 1 week. Hair was removed by using depilatory cream to observe the pink skin. Then, animals were randomly divided into groups (n=5) randomly to investigate hair regrowth. Minoxidil was topically applied daily as a positive control. Other treatments were given subcutaneously. All work with mice was in accordance with IACUC. Cell treatment dose: 1.0×10⁶ cells in 200 μL PBS were subcutaneously injected into 10 spots (20 μL per site) on the left side of the dorsal skin. Exosome treatment dose: 2.0×10⁹ exosomes in 200 μL PBS were subcutaneously injected to 10 spots (20 μL per site) on the left side of the backs. Negative control, miR-218-5p mimics and inhibitors (Sigma-Aldrich): administered according to the protocol provided by in vivo-jetPEI™ (Polyplus transfection). In brief, mice received ten injections per depilated dorsal skin. 400 ng miRNA mimic or inhibitor was dissolved in 90 μl 5% glucose (w/v) solution, and then 10 μl in vivo-jet PEI solution was added. Vortex-mix the solution immediately and incubate for 15 minutes at room temperature before injection. Control groups were injected with mimic control.

Skin tissue histology. Mice were euthanized and the whole dorsal skin was removed. Skin tissue was fixed in 4% paraformaldehyde and then cut into 5 μm thick sections by using Cryostat. Hematoxylin and eosin (H&E) and Masson trichrome were stained according to protocols. For immunofluorescent histochemistry, the primary antibodies used: mouse anti-β-catenin (ABCAM, ab6301) and rabbit anti-CD133 antibody (ab19898), rabbit anti-Ki67 antibody (ab16667) and rabbit SFRP2 (ab137560). The secondary antibodies used: goat anti-mouse IgG (Alexa Fluor® 488) (ab150113), goat anti-rabbit IgG (Alexa Fluor® 488) (ab150077) and goat anti-mouse IgG (Alexa Fluor® 555) (ab150114). Western blot. Samples were loaded and compared to a standard ladder (Precision Plus Protein™ Standards, Bio-Rad). Primary antibodies: mouse anti-β-catenin (ABCAM, ab6301), rabbit SFRP2 (ab137560), anti-Erk1 (pT202/pY204)+Erk2 (pT185/pY187) antibody [MAPK-YT] (ab50011), Anti-ERK1+ERK2 antibody (ab17942), and Anti-GAPDH antibody (HRP) (ab9482).

Statistical analysis. All quantitative experiments were done in triplicate unless otherwise indicated. Data were shown as mean±SEM and analyzed by two-tailed, unpaired Student's t test for comparison between the two groups. Data comparisons between more than two groups were analyzed using one-way analysis of variance (ANOVA) followed by the post hoc Bonferroni test. Data between grouped samples were analyzed by using two-way ANOVA. Differences with a P value less than 0.05 were considered statistically significant.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

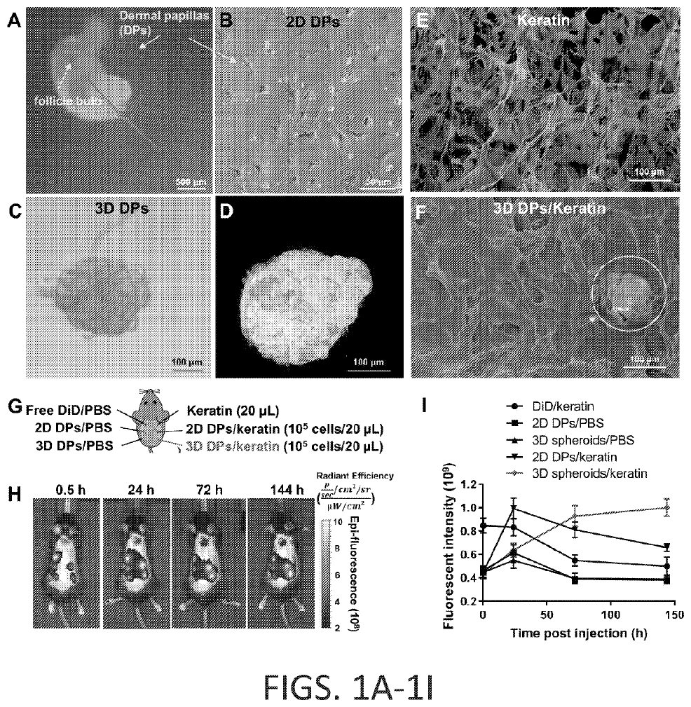

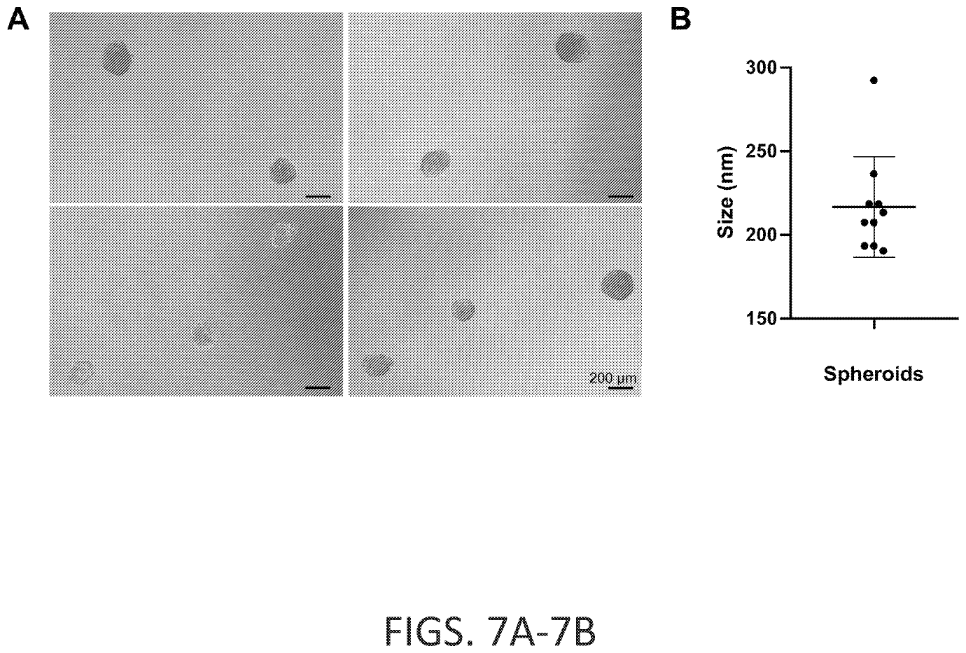

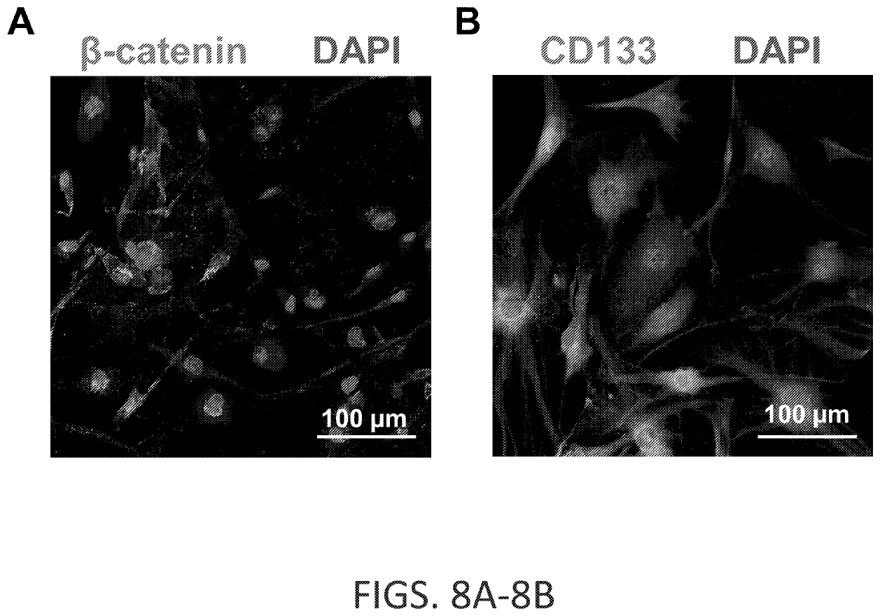

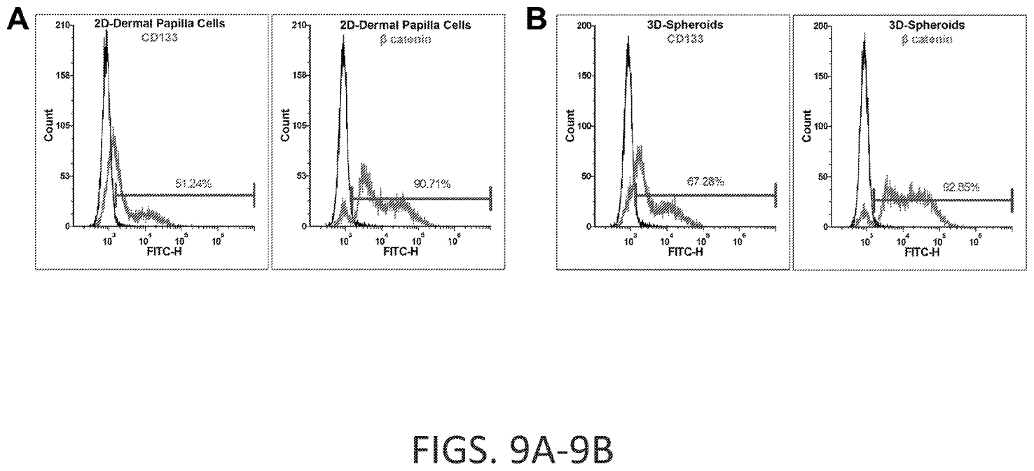

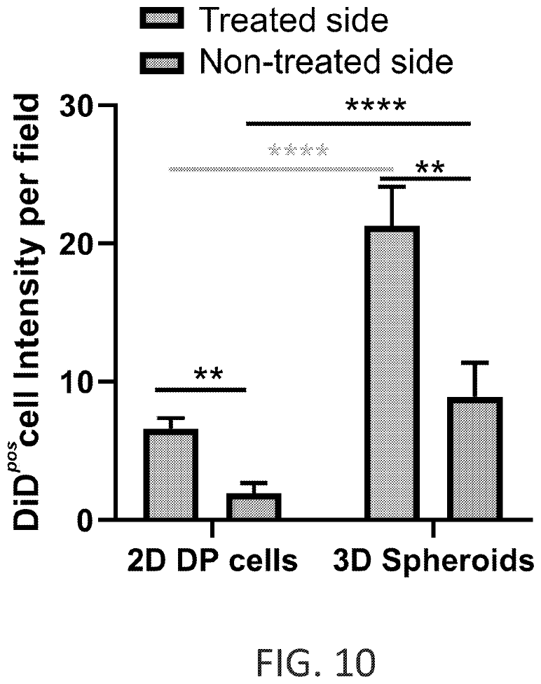

DP spheroids enhance expression of β-catenin and CD133 in vitro and transplantation engraftment in vivo. C57BL/6 mice are widely used in hair physiology studies because, after their backs are depilated, all hair follicles in the area enter the pause phase when mice come to seven-week old. DPs were isolated from the whiskers of C57BL/6 mice. Passages 3-6 were cultured and used in the present disclosure. FIG. 1A shows that DPs grew out from the hair follicle bulb and they exhibited a spindle-like shape when they formed multilayered parallel arrays. After passage, DPs displayed flattened, polygonal morphology (FIG. 1B). DP spheroids were formed by passaging DPs into low attachment flasks. The spheroids were 100-300 μm in diameter (FIG. 1C; FIG. 7) and expressed strong immunofluorescent signals of CD133 (green) and β-catenin (red) (FIG. 1D). The expression of CD133 and β-catenin was lower in two dimensional (2D) cultured DPs (FIG. 8). In spheroids, O-catenin expression was enhanced due to the increased cell-cell contact. CD133 expression was also analyzed and was enhanced in spheroids. CD133 positive DP cells exhibited hair inducing capacity in vivo. This was further confirmed via flow cytometry. Enhanced signal intensity was demonstrated in the gated cells from spheroids (FIG. 9).

To compare cell survival rate after transplantation, DPs and DP spheroids stained with DiD (1,1′-Dioctadecyl-3,3,3′,3′-Tetramethylindodicarbocyanine, 4-Chlorobenzenesulfonate Salt) were injected into the depilated backs of C57BL/6 mice. Hair keratins have been studied as cell culture scaffolds in vitro and in vivo as they are autologous, easy to extract, degradable and biocompatible. The viability of the DPs was preserved during encapsulation and maintained in the porous microarchitecture of keratin hydrogels. As shown in FIGS. 1E-1F, networks of keratin hydrogels and spheroids self-assemble inside hydrogels. 2D cells or 3D spheroids were collected and distributed in PBS or keratin hydrogel (10⁵ cells/20 μL) for subcutaneous administration. To improve the cell survival rate after transplantation, DPs/PBS, DPs/keratin, DP spheroids/PBS, and DP spheroids/keratin were compared (FIG. 1G). IVIS demonstrated that spheroids demonstrated an enhanced retention and survival rate after injection (FIGS. 1F-1I). Keratin further supported cell attachment and proliferation. DP spheroids/keratin hydrogel maintained high cell viability after engraftment onto the dorsal skin of mice.

### Example 2

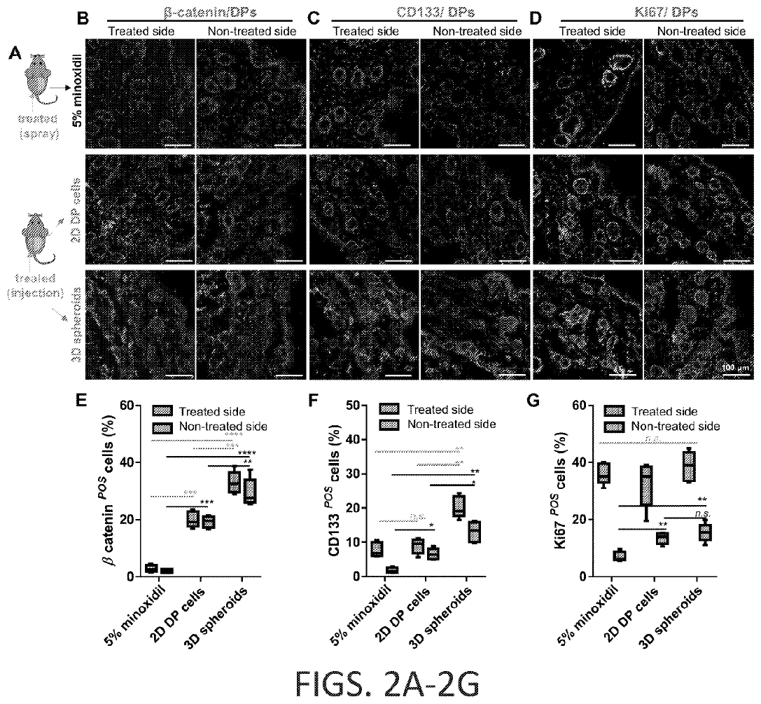

DP spheroids enhance the expressions of β-catenin, CD133 and Ki67 in hair follicles in vivo. DPs and DP spheroids were injected into one side of mice after depilation (ten one-time injections, evenly distributed on the treated side, 10⁵ cells in 20 μL per spot) or 5% minoxidil was topically treated on the treated side daily (FIG. 2A). Ten days later, dorsal skin samples were taken from both the injected sites (left) and non-treated sites (right).). Ten days later, both the injected site (left) and remote site (right) of back skins were collected. β-catenin, CD133, and Ki67 were stained (FIGS. 2B-2D) to compare the hair follicle-inducing mechanisms of DPs, DP spheroids, and 5% minoxidil. Corresponding quantification results were shown in FIGS. 2E-2G.

Minoxidil exerts a vasodilator effect on hair follicles, leading directly to the proliferation of follicle cells. On the treated side, 5% minoxidil promoted the expression of Ki67 to 37.2% on day 10, but not β-catenin (2.3%) nor CD133 (7.2%), on the treated side, which suggests that the proliferation and growth of whole hair follicle cells were promoted non-specifically. In contrast, the injection of 2D DPs enhanced the expression of β-catenin (19.1%) and CD133 (9.8%) on the treated side, β-catenin (19.9%) and CD133 (6.8%) on the non-treated side. The injection of 3D DP spheroids enhanced the expression of β-catenin (32.6%) and CD133 (19.1%) on the treated side, β-catenin (27.8%) and CD133 (13.7%) on the non-treated side. Both showed a certain amount of cell migration on the non-treated site and enhanced expressions of β-catenin, CD133, and Ki67 on the non-treated side compared to minoxidil. In particular, DP spheroids showed much stronger β-catenin and CD133 staining compared to the DPs group. Minoxidil, however, did not alter B-catenin or CD133 expression on both sides. The Ki67 signals on the treated side were 37.2%, 37.4% and 39.5% for minoxidil, 2D DPs and 3D DPs respectively with no significant difference, however, the Ki67 signals on the non-treated side were 7.1%, 12.6%, 17.5% respectively. Cell migration and viability (red signals) might contribute to this remote promotion. This study demonstrated that implanted cells not only influence the hair follicles at the injected site but also regulate the hair follicles at a non-treated area. DP spheroids engraftment was more effectively in regulating hair follicle growth. In addition, because the migration of cells in the skin was quite limited, thus paracrine effects could also be an important mechanism in this process.

### Example 3

DP spheroid treatment accelerates hair regrowth in C57BL/6 mice. C57BL/6 mice are widely used in hair physiology studies because all hair follicles enter the pause stage after depilation at around 7 weeks. Eight-week-old male C57BL/6 mice were used in the present disclosure. The skin was pink after hair epilated, and increasingly darker during the treatment. Minoxidil, the gold standard treatment for hair loss, was used as the positive control. Cultured DPs were reported to lose their hair inductive capacity gradually. Given the limited effects of 2D-cultured DPs administration (see, e.g., FIG. 2), the hair follicle induction capacity of 3D spheroids was further investigated.

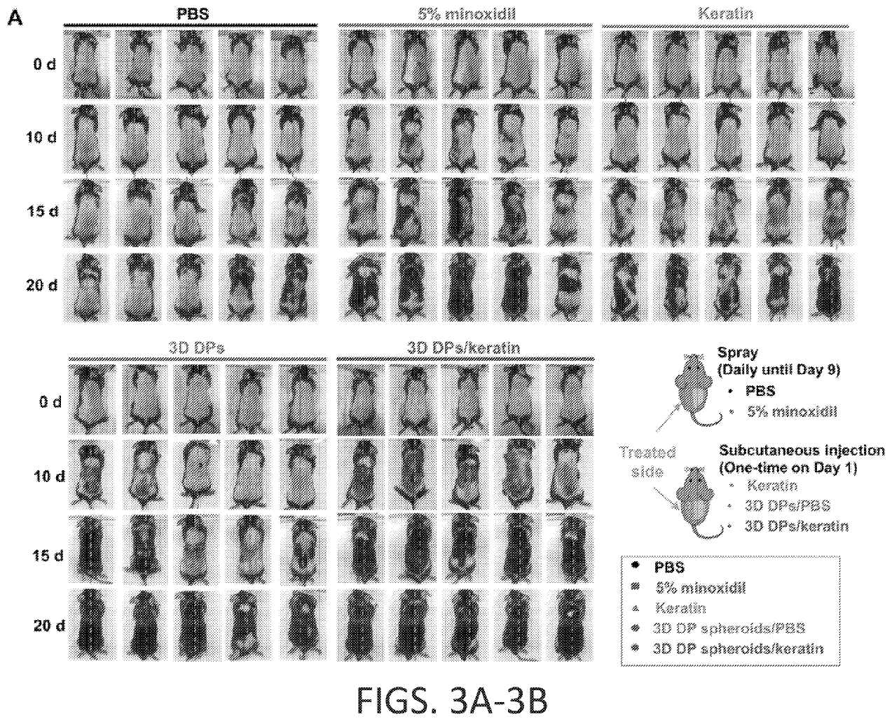

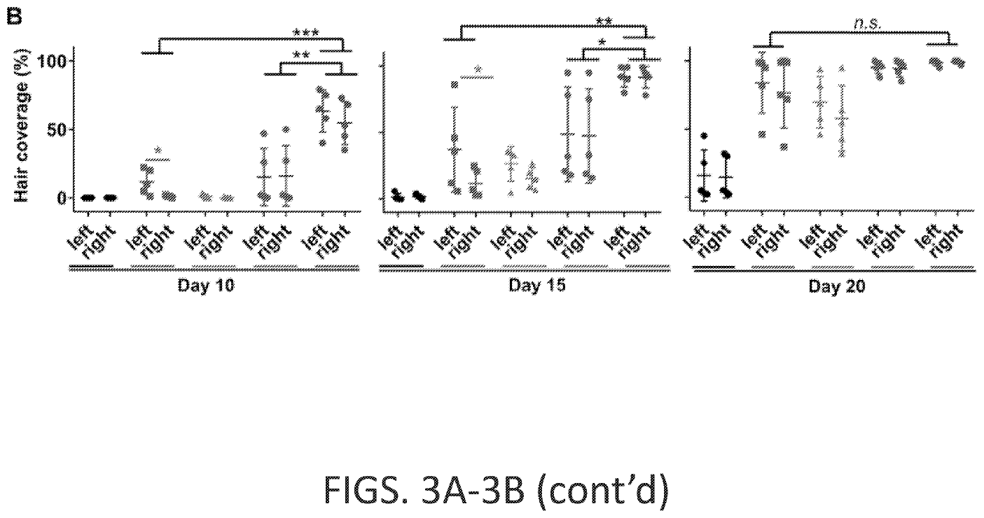

The state of hair re-growth in depilated mice was photographed on days 0, 10, 15, and 20, and hair growth area was analyzed via morphological observation (FIGS. 3A-3D). On day 10, black pigmentation started to show in the minoxidil, and cell treated groups. All mice in the DP spheroids/keratin group showed a much darker skin than other groups. On day 15, the treated side of the minoxidil group showed 35% fur coverage and 10% for the untreated side. The DP spheroids/PBS group showed an average of 40% fur coverage and the fur coverage of DP spheroids/keratin was approximately 90%. The engraftment and survival rate of cells in PBS was uneven and so was the resultant therapeutic effect, while keratin enhanced the overall spheroids' engraftment and cell survival rate.

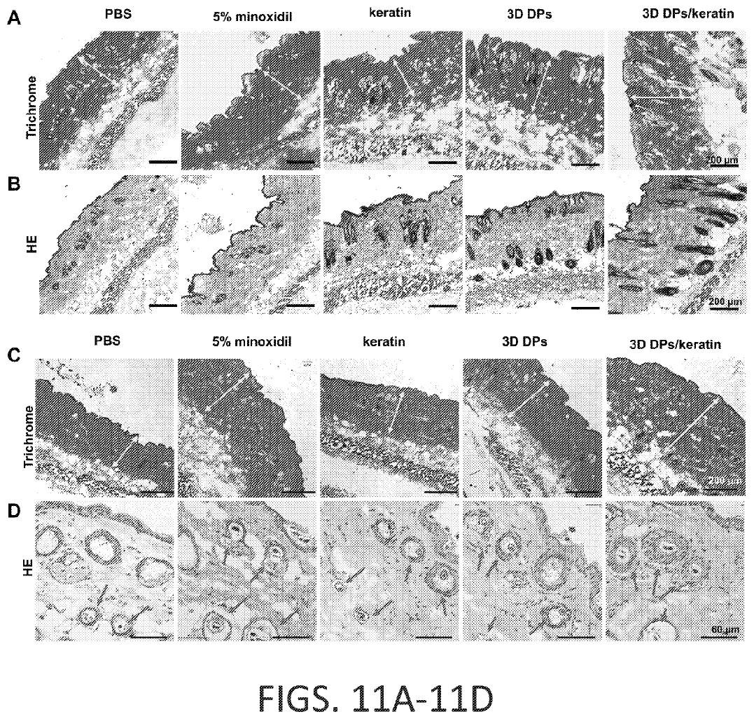

Masson trichrome and H&E staining demonstrated that the DP spheroids/keratin treatment group resulted in larger hair follicles and more collagen distribution compared to other groups (FIG. 11A-11B). The transition of the resting follicles into anagen phase lead to an increased follicle size. In contrast, the control group had smaller follicles and thinner collagen layer compared to the group treated with DP spheroids. The injection of DP spheroids did not induce inflammation towards the skin tissue since those cells were autologous. In addition, the keratin hydrogels did not evoke an acute immune response in vivo. The thinnest fur area (without visible hair; FIG. 11C) was collected and the thickest fur area (with hair, FIG. 11D) of each group. The deeper and thicker fair follicles in 3D spheroid groups in FIG. 1C are evident. In the PBS group, even though there were hair growth, only superficial hair follicles entered the anagen cycle (FIG. 11D).

### Example 4

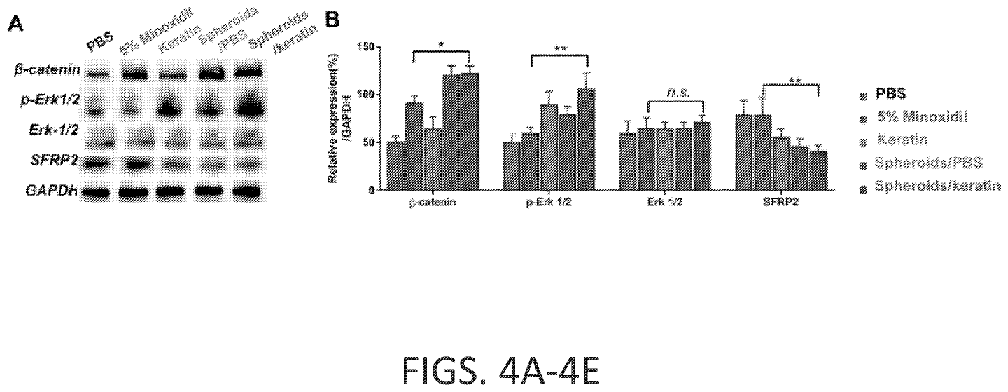

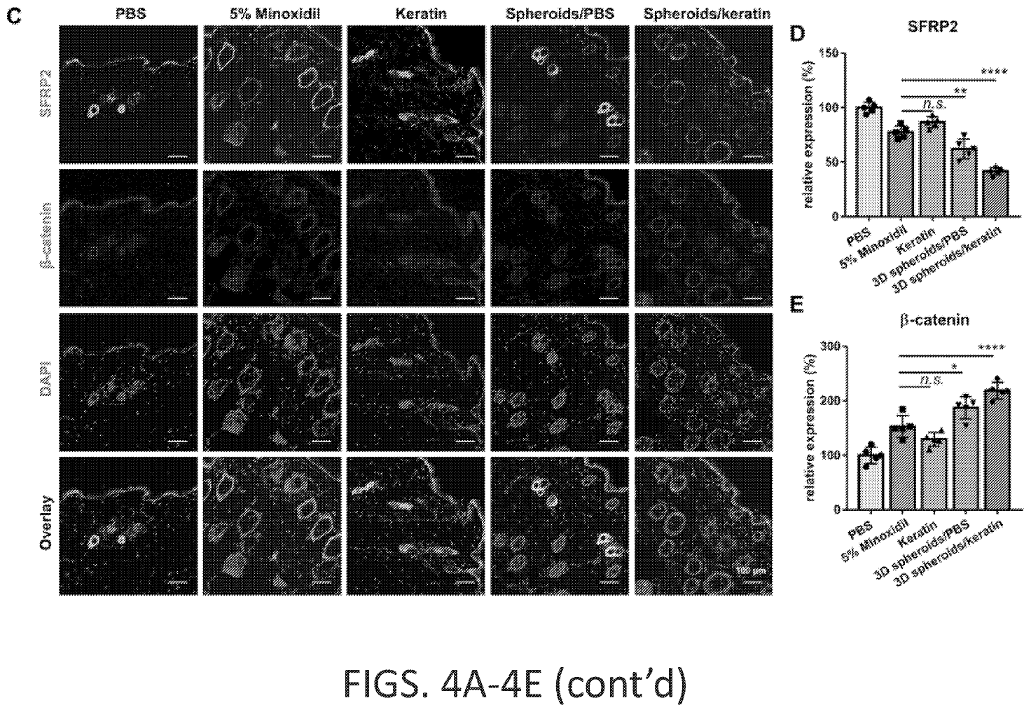

DP spheroids accelerate the onset of hair follicle anagen by upregulating p-catenin. To elucidate the molecular mechanism, protein levels of β-catenin, extracellular signal-regulated protein kinases 1 and 2 (Erk^{1/2}), phospho-ERK1/2 (p-Erk^{1/2}), the secreted frizzled related protein 2 (SFRP2), and glyceraldehyde 3-phosphate dehydrogenase (GAPDH) were analyzed in the depilated dorsal skin 20 days after treatment by Western blot (FIGS. 4A-4B). The results showed that the expression of β-catenin and p-Erk^{1/2} in DP spheroids groups was up-regulated and that of SFRP2 was down-regulated in comparison to the control and minoxidil group. WNT/β-catenin pathway has been demonstrated its critical role in regulating the hair cycle, promoting hair growth. β-catenin is a key regulator of hair follicle growth, and it is reported to be the primary initiator of the anagen phase. SFRP2 has been reported to be functional in several cellular activities and biological processes via down-regulating β-catenin in nuclear translocation. These results indicated that DP spheroids may activate hair follicle development through upregulating β-catenin signaling in the WNT pathway. Immunofluorescent staining of SFRP2 and β-catenin (FIGS. 4C-4E) was also performed to further confirm the downregulation of SFRP2 and upregulation of β-catenin in spheroids/keratin group. Thus, DP spheroids culture could be an effective strategy to improve DP therapeutic potency before cell administration for hair loss treatment.

### Example 5

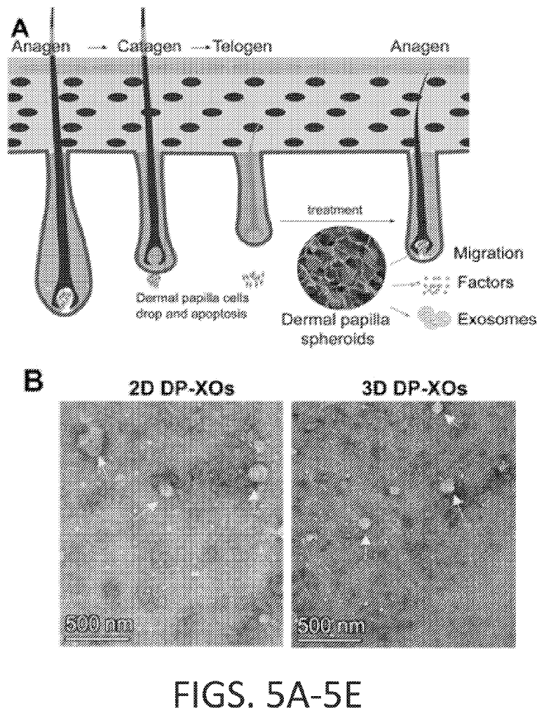

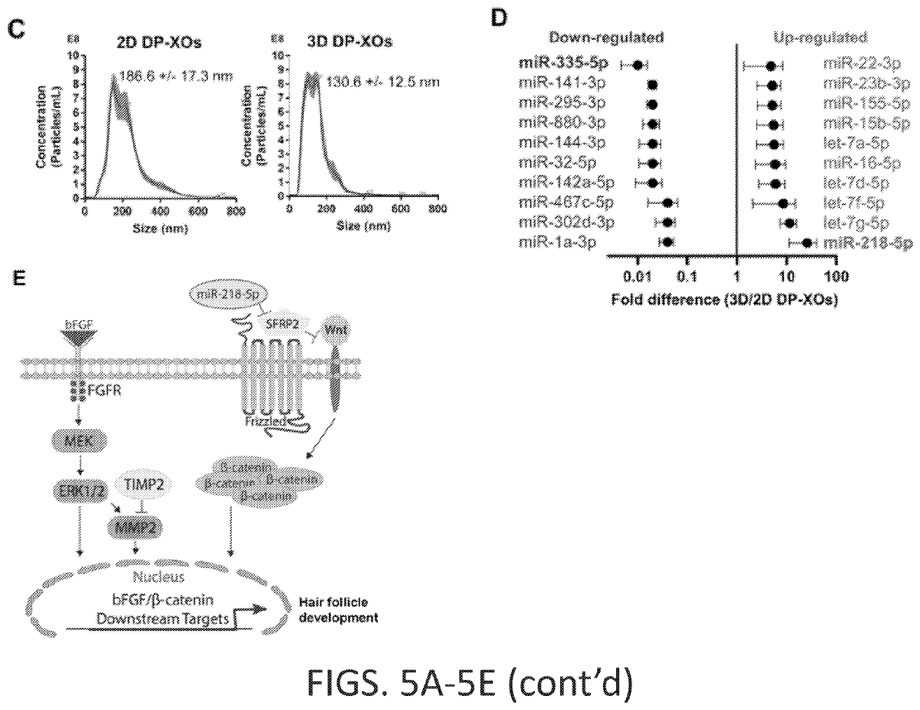

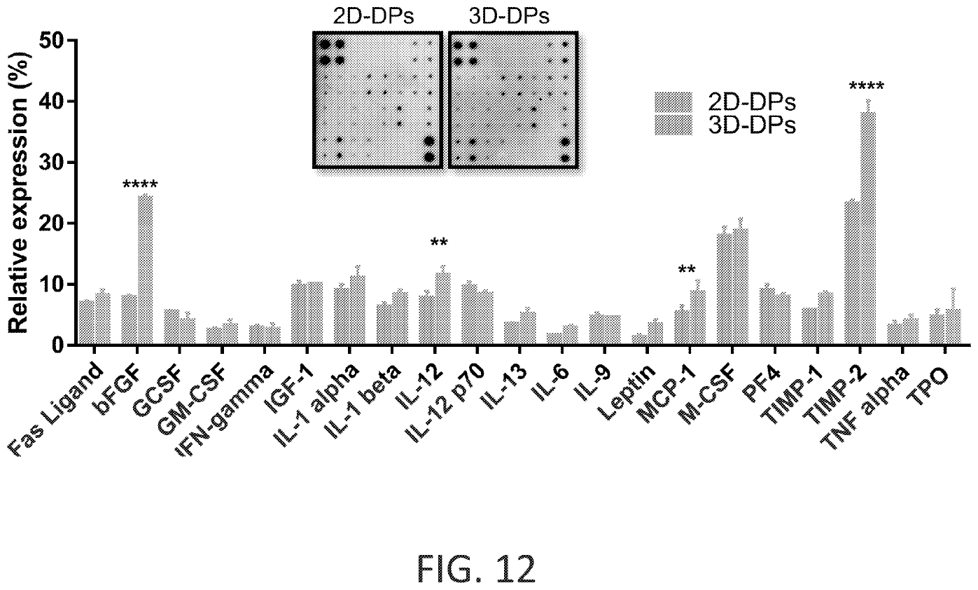

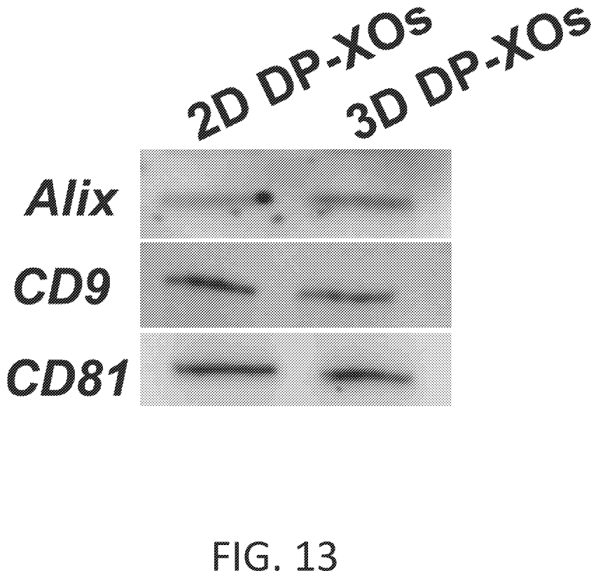

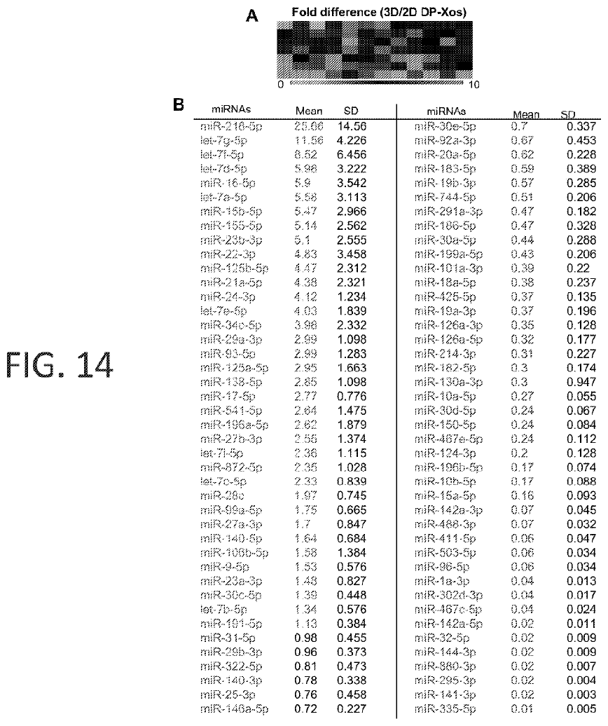

Comparison of protein and microRNA profiles between DPs and DP spheroids. DP spheroids not only affected the local injected site, but also affected the regulation of the hair follicle cycle in a remote area due to paracrine signaling. DPs stimulate follicular development and modulate mesenchymal-epithelial interactions by releasing various growth factors and exosomes (FIG. 5A). Exosomes and conditioned media from DPs and DP spheroids were isolated and detected. Compared to the other treatment groups, spheroid secretome resulted in significantly higher expression levels of bFGF and TIMP2 (FIG. 12). Transmission electron microscopy (TEM, FEI Talos F200X) images showed the shape of exomes (FIG. 5B). The average size of DPs derived exosomes (DP-XOs) was around 180 nm and the average size of DP spheroid derived exosomes (DP spheroid-XOs) was around 130 nm (FIG. 4C). FIG. 13 indicated the exosomal makers, Alix, CD9, and CD81. To compare miRNAs in the exosomes, an miRNA array was performed and results indicated that DP spheroid-XOs expressed miR-218-5p at significantly higher levels than DP-XOs (FIG. 5D and FIG. 14).

As shown in FIG. 5E, bFGF can be accepted by FGFR on cell membrane-bound and upregulate the expression of p-Erk1/2, thus regulate the expression of β-catenin. TIMP2 was known to inhibit the expression of MMP2, which might hinder the migration and proliferation of DPs. Growth factors are involved in the promotion of hair growth cycles and the mechanism of bFGF has been demonstrated before. Although bFGF and TIMP2 were higher in spheroid secretome, they were about doubled compared to 2D cell secretome; while, miR-218-5p was enriched 25-fold.

### Example 6

DP spheroid-derived exosome treatment for hair regrowth. Cell-based therapy is a promising step forward in the pursuit of achieving hair follicle neogenesis. However, the harnessing of cellular components through to be the therapeutic catalyst would make for an even more convenient therapeutic product, obviating the cell as the therapeutic carrier. Both exosomes and secretome might contribute to the hair grow process, differentially expressed miR-218-5p in exosomes might contributes to the higher efficacy of 3D spheroids in promoting hair growth compared to 2D DPs. Also, according literatures, miR-218-5p directly targets SFRP2, and thus it upregulates the WNT/β-catenin pathway. Thus, miR-218-5p up-regulated exosomes were the main research object in this study.

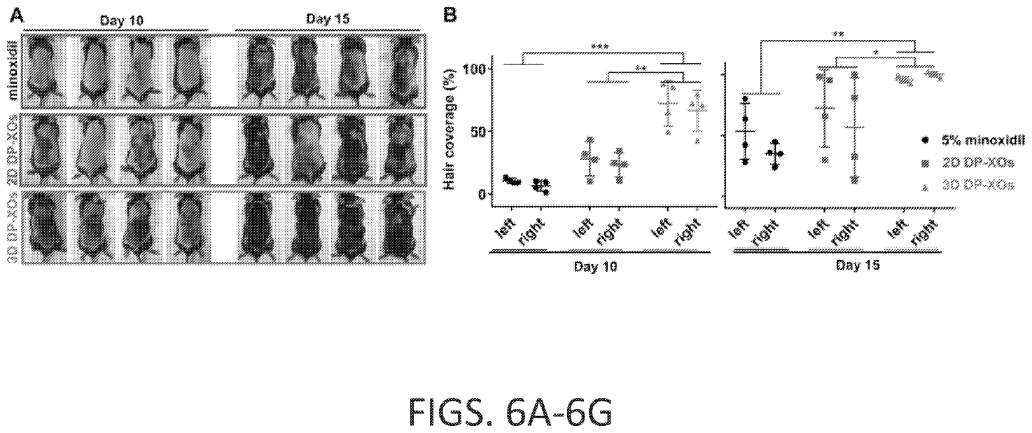

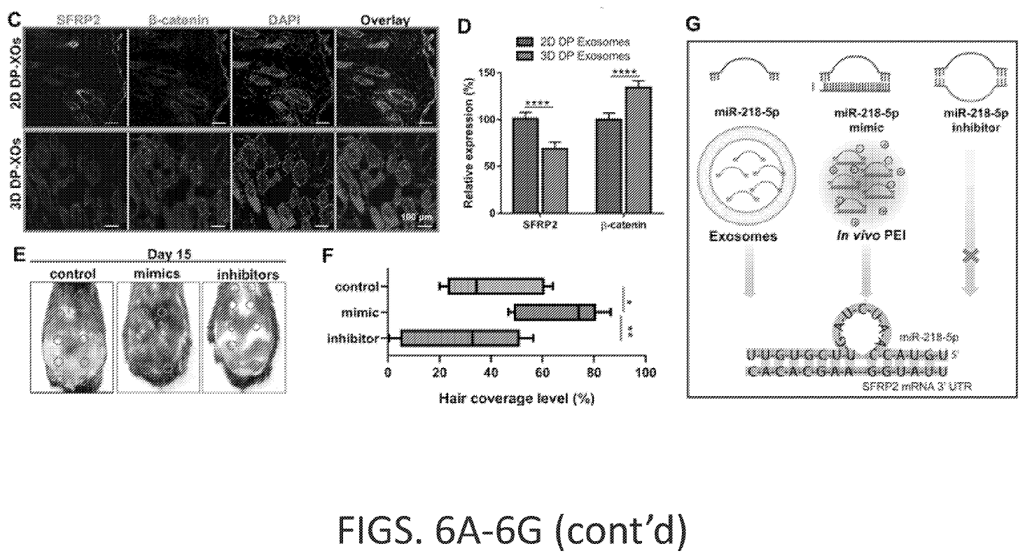

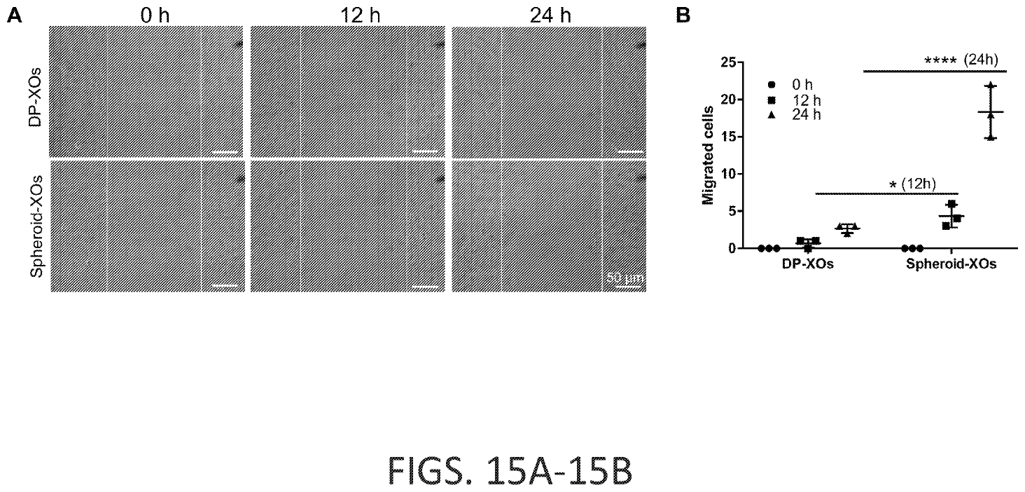

Results of experiments demonstrated that exosomes from spheroids effectually promote DP migration compared to exosomes from 2D cells (FIG. 15), thus it had the potential to promote DP migration to the follicle bulb. Then, in vivo experiments were performed on C57BL/6 mice. Exosomes were administrated to one side of the back skin (FIG. 2A) and compared to 5% minoxidil topical treatment. In morphological observation (FIG. 6A) and corresponding quantification (FIG. 6B), the therapeutic efficacy of 2D DP-XOs did not show significant difference from minoxidil groups, while 3D DP spheroid-XOs accelerated hair growth and the effect was comparable to cell treatment. To explore the changes of SFRP2 and β-catenin expression in skin after treatment, dorsal skin was harvested from different groups on Day 15. Immunostaining confirms that β-catenin expression was increased in DP spheroids-XOs group when compared to those treated with 2D DP-XOs and minoxidil. DP spheroid-XOs can regulate hair growth cycles by downregulating SFRP2 and upregulating β-catenin (FIGS. 6C-6D). Since miR-218-5p directly targets SFRP2, miR-218-5p mimics and inhibitors were used to further validate the importance of miR-218-5p enrichment.

### Example 7

miR-218-5p plays a crucial role in exosome-mediated hair regrowth. One of the main challenges in miRNA treatment is delivery. The in vitro model does not accurately reflect whether miR-218-5p plays an important role in regulating hair follicle growth considering the diverse types of cells in the follicles. Herein, a mmu-miR-218-5p mimic was mixed with PEI (in vivo-jetPEI™; Polyplus Transfection, Illkirch, France) according to the manufacturer's instructions. The PEI/negative control, PEI/mimics or PEI/inhibitors polyplex solution (10 μL per site) was carefully administered subcutaneously into the dorsal skin of depilated mice (FIG. 6E). By targeting SFRP2, miR-218-5p mimics promoted the hair follicle development, while miR-218-5p inhibitors inhibited the onset of the anagen phase in the hair follicle cycle. Significant hair regrowth effects were demonstrated with the treatment of miR-218-5p mimic, as compared to the control group and the miR-218-5p inhibitors. However, such effects (50˜90% hair coverage) were less potent than those from exosome treatment (95˜100% hair coverage) on day 20. These data suggest that exosomes contain a variety of miRNAs and proteins. miR-218-5p is only one that is involved in promoting hair growth.

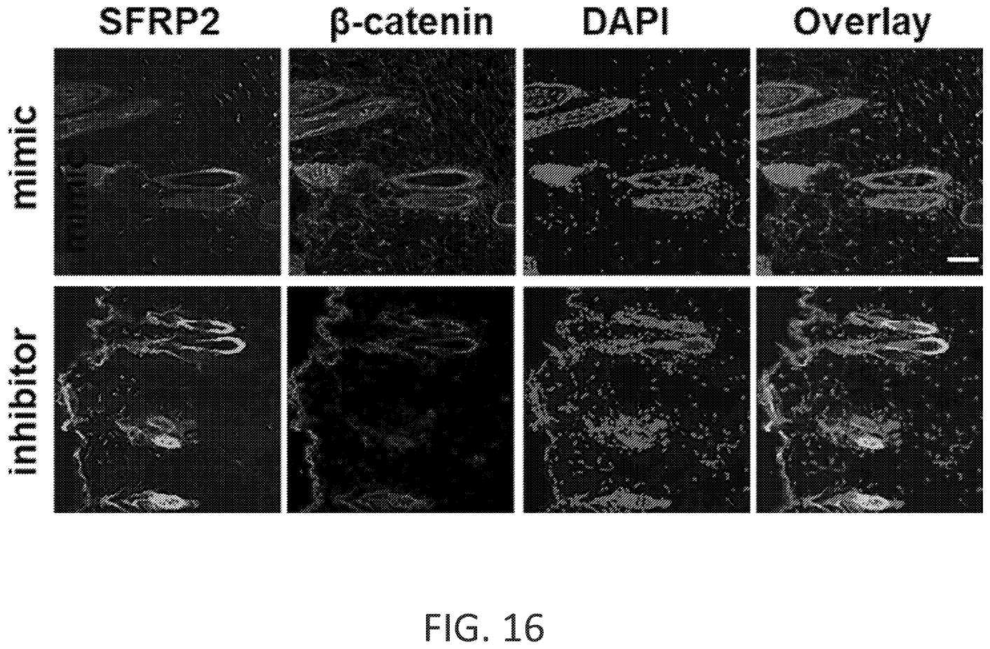

Various different strategies were used to promote hair growth. As illustrated in FIG. 6G, both miR-218-5p enriched exosomes and miR-218-5p mimic encapsulated by in vivo PEI promoted the transcript of miR-218-5p. The genetic structure of miR-218-5p showed that it would target SFRP2 directly. To demonstrate the regulatory effects of miR-218-5p and Wnt signaling in the hair growth cycle, transcriptional mediators, SFRP2 and β-catenin were examined by Western blot. Skin samples (day 15) showed that miR-218-5p mimics robustly increased endogenous β-catenin expression, while treatment with miR-218-5p inhibitors showed a decreased β-catenin expression (FIG. 16).

Together these data support the main mechanism of stimulation of Wnt signaling by miR-218-5p enriched exosomes through down-regulation of inhibitors of Wnt signaling SFRP2, and upregulation of β-catenin.

1. A composition comprising a plurality of exosomes derived from human dermal papilla cell (DP) spheroids, wherein the plurality of exosomes comprise at least one of:
(i) increased expression of at least one miRNA; and/or
(ii) decreased expression of at least one miRNA.

2. The composition of claim 1, wherein the plurality of exosomes are derived from DP spheroids cultured using three-dimensional (3D) cell culture.

3. The composition of claim 1, wherein the plurality of exosomes comprise at least one of (i) and/or (ii) as compared to a naturally occurring DP-derived exosome.

4. The composition of claim 1, wherein the plurality of exosomes comprise at least one of (i) and/or (ii) as compared to a DP-derived exosome cultured using two-dimensional (2D) cell culture.

5. The composition of claim 1, wherein the at least one miRNA of (i) is selected from the group consisting of miR-218-5p, let-7f-5p, let-7g-5p, let-7d-5p, miR-16-5p, let-7a-5p, miR15b-5p, miR-155-5p, miR-23b-3p, miR-22-3p, miR-125b-5p, miR-21a-5p, miR-24-3p, let-7e-5p, miR-34c-5p, miR-29a-3p, miR-93-5p, miR-125a-5p, miR-138-5p, miR-17-5p, miR-541-5p, miR-196a-5p, miR-27b-3p, let-7i-5p, miR-872-5p, let-7c-5p, miR-28c, miR-99a-5p, miR-27a-3p, miR-140-5p, miR-106b-5p, miR-9-5p, miR-23a-3p, miR-30c-5p, let-7b-5p, miR-191-5p, or any combinations thereof.

6. The composition of claim 1, wherein the at least one miRNA of (i) is increased in the plurality of exosomes by at least 1.1-fold.

7. The composition of claim 1, wherein the at least one miRNA of (i) is selected from the group consisting of miR-218-5p, let-7f-5p, let-7g-5p, or any combinations thereof.

8. The composition of claim 1, wherein the at least one miRNA of (ii) is selected from the group consisting of miR-31-5p, miR-29b-3p, miR-322-5p, miR-140-3p, miR-25-3p, miR-146a-5p, miR-30e-5p, miR-92a-3p, miR-20a-5p, miR-183-5p, miR-19b-3p, miR-744-5p, miR-291a-3p, miR-186-5p, miR-30a-5p, miR-199a-5p, miR-101a-3p, miR-18a-5p, miR-425-5p, miR-19a-3p, miR-126a-3p, miR-126a-5p, miR-214-3p, miR-182-5p, miR-130a-3p, miR-10a-5p, miR-30d-5p, miR-150-5p, miR-467e-5p, miR-124-3p, miR-196b-5p, miR-10b-5p, miR-15a-5p, miR-142a-3p, miR-488-3p, miR-411-5p, miR-503-5p, miR-96-5p, miR-1a-3p, miR-302d-3p, miR-467c-5p, miR-142a-5p, miR-32-5p, miR-144-3p, miR-880-3p, miR-295-3p, miR-141-3p, miR-335-5p, or any combinations thereof.

9. The composition of claim 1, wherein the at least one miRNA of (ii) is reduced in the plurality of exosomes by at least 0.9-fold.

10. The composition of claim 1, wherein the at least one miRNA of (ii) is selected from the group consisting of miR-335-5p, miR-141-3p, miR-295-3p, miR-880-3p, miR-144-3p, miR-32-5p, miR-142a-5p, or any combinations thereof.

11. The composition of claim 1, wherein the plurality of exosomes further comprise increased expression of at least one hair follicle regulatory gene selected from FGF2, TIMP2, or a combination thereof.

12. (canceled)

13. A method of generating a plurality of exosomes capable of modulating at least one characteristic of skin tissue, the method comprising:
culturing human dermal papilla cell (DP) spheroids using three-dimensional (3D) cell culture; and
isolating a plurality of exosomes from the DP spheroids.

14. The method of claim 13, wherein the plurality of exosomes comprise at least one of:
(i) increased expression of at least one miRNA; and/or
(ii) decreased expression of at least one miRNA

15-16. (canceled)

17. The method of claim 13, wherein the at least one miRNA of (i) is selected from the group consisting of miR-218-5p, let-7f-5p, and let-7g-5p, or any combinations thereof.

18. The method of claim 13, wherein the at least one miRNA of (ii) is selected from the group consisting of miR-335-5p, miR-141-3p, miR-295-3p, miR-880-3p, miR-144-3p, miR-32-5p, miR-142a-5p, or any combinations thereof.

19-20. (canceled)

21. A method of treating a skin condition or disease, the method comprising:
administering a plurality of exosomes derived from dermal papilla cell (DP) spheroids to a subject in need thereof;
wherein administering the plurality of exosomes modulates at least one characteristic of the subject's skin tissue.

22-25. (canceled)

26. The method of claim 21, wherein the at least one miRNA of (i) is selected from the group consisting of miR-218-5p, let-7f-5p, and let-7g-5p, or any combinations thereof.

27. The method of claim 21, wherein the at least one miRNA of (ii) is selected from the group consisting of miR-335-5p, miR-141-3p, miR-295-3p, miR-880-3p, miR-144-3p, miR-32-5p, miR-142a-5p, or any combinations thereof.

28-30. (canceled)

31. The method of claim 21, wherein modulating at least one characteristic of the subject's skin tissue comprises at least one of:
(i) increasing expression of β-catenin;
(ii) decreasing expression of SFRP2;
(iii) enhancing DP cell migration;
(iv) enhancing hair follicle growth; and/or
(v) maintaining anagen phase of hair cycle; and
wherein modulating at least one of (i)-(v) treats the subject's skin condition or disease.

32. (canceled)

33. The method of claim 21, wherein the skin condition or disease comprises male or female-pattern hair loss, alopecia areata, telogen effluvium or anagen effluvium.
